(19) United States
(12) Patent Application Publication
(10) Pub. No.: US 2004/0039042 A1
(43) Pub. Date: Feb. 26, 2004

(54) Method of transdermal drug delivery

(76) Inventor: Thomas E. Fleming, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/226,730

(22) Filed: Aug. 23, 2002

(51) Int. Cl. A61K 31/415; A61K 9/70

(52) U.S. Cl. 514/406

(57) ABSTRACT

The present invention relates to a method for transdermal drug delivery of insoluble drugs, especially those with severe side effects. It also relates to a new use for the drug Carafate®. Specifically, the present invention describes a method for delivering insoluble drugs transdermally by making a suspension of the powdered form of the drug in an isotonic solution. Drugs which can be delivered by this method include nonsteroidal anti-inflammatory drugs (NSAIDs), Vioxx®, Celebrex®, Accutane®, and Carafate®. The invention also describes a solution of a powdered form of Carafate® in an isotonic solution, which can then be used to treat conditions such as acne, herpes simplex II cold sores, root canal wounds, and dry sockets after teeth have been removed.

### BACKGROUND OF THE INVENTION

This invention relates generally to a method for transdermal drug delivery whereby a drug is put into an isotonic solution and rubbed into the skin.

Many of the drug treatments available today cause severe side effects. There is a great need for a method to deliver these drugs which will reduce or eliminate the side effects which prohibit many patients from using drugs which may help them. The present invention describes such a method.

Although topical treatments for diseases of the skin have been used for centuries, transdermal drug delivery was not explored until recently because the scientific community believed that the skin was a dead, impenetrable layer. This belief has been overcome in recent years, and transdermal drug delivery methods using vehicles such as creams, lotions, powders, and gels have been used for soluble drugs. Insoluble drugs, however, have not been used in transdermal drug delivery methods. The current invention teaches a method to deliver soluble and insoluble pharmaceutical agents through the skin, especially those which cause severe gastrointestinal (GI) problems, by combining a powdered form of one or more pharmaceutical agents with an isotonic solution such as Ocean®, and rubbing the mixture into the skin. The powdered form of the pharmaceutical agents must be combined with an isotonic solution because powders do not adhere well to the skin.

The prior art teaches methods of transdermal drug delivery for soluble drugs only. W.O. Patent No. 02/45701, a method to prevent crystallization of supersaturated solutions, teaches the use of only soluble active agents for transdermal delivery. W.O. Patent No. 87/02870 teaches a nicotine patch in which the vapors are absorbed by the skin. E.P. 0455396A1 teaches the use of an isotonic gel for delivery of soluble drugs to reduce any burning sensation associated with delivery of a drug. Solvents commonly used for transdermal drug delivery include water, alcohol, glycerin, and propylene glycol. (Fitzpatrick's Dermatology in General Medicine, Fifth Edition, p. 2710). The prior art does not teach transdermal delivery of insoluble drugs, nor does it teach the delivery of drugs in an isotonic aqueous solution. In fact, the prior art teaches away from the transdermal delivery of insoluble drugs.

Recently the New York Times published an article entitled “More Than the Patch: New Ways to Take Medicine Via the Skin” (Jul. 2, 2002) on new methods of transdermal drug delivery. According to the article, large and/or insoluble molecules cannot be delivered transdermally. Therefore, scientists are working on methods which involve delivering electric shocks to the skin to open the pores, or patches which contain multiple tiny needles which can penetrate the stratum corneum to deliver drugs. The idea of applying and delivering insoluble drugs transdermally using an isotonic solution has consistently been overlooked. Transdermal delivery of drugs to the target tissue as described by this invention is much needed because it is an effective means of drug delivery which does not include side effects associated with oral delivery of the drugs.

### SUMMARY OF THE INVENTION

It is an object of the present invention to give a method for the transdermal delivery of drugs, both soluble and non-soluble.

It is another object of the present invention to provide a safe and effective delivery method for drugs which cause toxicity and side effects in patients.

It is another objective of the present invention to provide an alternate method of drug delivery for those drugs which produce gastrointestinal toxicity.

It is another object of the present invention to deliver the needed drugs directly to the target tissue, rather than systemically.

It is yet another object of the present invention to reduce dosages in some cases, thereby reducing effects the drugs may have on the kidneys or liver.

It is another object of the present invention to provide a delivery method for drugs which consists of placing a powdered form of the drug in an isotonic solution such as Ocean®, and rubbing an effective amount of the mixture on the target location of skin.

It is yet another objective of the present invention to deliver NSAIDs transdermally.

It is yet another object of the present invention to provide a delivery method for drugs including Accutane®, Vioxx®, Celebrex®, and Carafate®.

It is yet another object of the present invention to describe new uses for the drug Carafate®, including but not limited to anti-inflammatory, antibiotic, and antiviral.

It is yet another object of the present invention to use the method of transdermal drug delivery for application to wounds from root canals, dry sockets after a tooth has been removed, herpes simplex 11 cold sores, and acne.

### DETAILED DESCRIPTION OF THE INVENTION

Hereinafter, Ocean® is used to refer to any aqueous isotonic solution (Fleming & Company, Fenton, Mo.). Other isotonic solutions include 0.9% sodium chloride solution, sodium citrate solution, or any other physiological solution.

A drug must penetrate the layers of the skin to reach the underlying tissue to be delivered transdermally. First, the drug must penetrate the stratum corneum, then it must diffuse through the viable epidermis into the dermis, where it can enter the vascular system, or it can continue through the dermis and hypodermis into the underlying tissues. (Fitzpatrick's, p. 2700)

The stratum corneum is the limiting factor in the transdermal delivery of substances. It is a highly organized, differentiated layer approximately 10-20 μm thick, containing bundles of water-insoluble proteins embedded in intercellular lipid, the combination of which serves as an effective skin barrier. The proteins of the stratum corneum are associated with corneocytes, which contain a core of keratins surrounded by an envelope of cross-linked proteins. The intercellular lipids are organized in sheets, creating a barrier to diffusion across the stratum corneum. The rate of diffusion of these lipids is much lower than that of cellular membranes, reducing the rate of diffusion across the lipid barrier. Despite the organization and structure of the stratum corneum, some molecules do get through. Appendages penetrate the stratum corneum and epidermis, which provide potential sites of penetration of the skin barrier. But, because the ratio of appendages to skin area is very low, it is thought that other methods are more important for molecules to penetrate the stratum corneum.

Research has shown that some molecules can pass through the lipid barrier and/or the inner lumen of the corneocytes to penetrate the stratum corneum. Corneocytes undergoing desquamation are fairly permeable, even to bulky molecules such as mercury. Other evidence for the penetration of corneocytes is the swelling of corneocytes when skin is immersed in water. This is readily visualized when one takes a bath, leaving fingers and toes wrinkled from the corneocytes which have absorbed water.

After a substance has made its way through the stratum corneum, it enters the viable epidermis. The viable epidermis is larger than the stratum corneum and less resistant to diffusion, resulting in a dilution of the substance. The viable epidermis has many metabolic activities, including drug-metabolizing enzymes which could further reduce the concentration of the drugs, affecting the availability of the drug to the underlying tissue. However, if a drug or other substance is not metabolized in the epidermis, it can easily diffuse into the dermis. Once in the dermis, the drug may undergo resorption, which is the uptake of compounds by the cutaneous microvasculature. The rate of resorption is related to the surface area of the capillaries as well as to the rate of blood flow. Resorption may limit delivery of the drug to the target site. Despite resorption, however, the present invention describes a variety of drugs, for example, Nonsteroidal anti-inflammatory drugs, Accutane®, and Carafate® to name a few, that do reach the underlying muscle, tissue, and joints by diffusing through the dermis and hypodermis, and successfully alleviate the symptoms related to certain diseases.

### Nonsteroidal Anti-Inflammatory Drugs (NSAIDs)

Nonsteroidal anti-inflammatory drugs (NSAIDs) are used for the treatment of arthritis because of their anti-inflammatory, analgesic, and antipyretic activities. NSAIDs function by inhibiting cyclooxygenase-2 (COX-2). COX-2 is expressed after stimulation with inflammatory cytokines, such as those present in arthritis. COX-2 is required for the production of prostaglandin at inflammatory sites, as well as PGl₂, a vasodilator and inhibitor of platelet aggregation. The commonly used NSAIDs include Vioxx®, Celebrex®, Anaprox, Indocin, Lodine, Motrin, Naprelan, Narposyn, Orudis, Oruvail, Relafen, Tolectin, Toradol, Trilisate, and Voltaren.

The blockade of cyclooxygenase also results in inhibition of caricinogenesis and subsequent development of tumors. COX-2 is induced in various carcinomas, indicating that COX-2 plays a key role in tumorigenesis, and research suggests that tumors produce COX-2 to support their own growth. Inhibiting COX-2 with NSAIDs, therefore, could be used for tumor prevention or tumor growth suppression.

NSAIDs, in addition to inhibiting COX-2, also inhibit COX-1. COX-1 is found in almost all tissue, especially in the gastric mucosa where it stimulates production of protective prostaglandins. For this reason, NSAIDs often cause severe gastrointestinal (GI) toxicity such as bleeding, ulceration, perforation of the stomach, small intestine, or large intestine, dyspepsia, and vomiting. These conditions can occur at any time, without warning, and are 10 times more likely to occur in individuals with a prior history of peptic ulcer disease or gastrointestinal bleeding. The rate of occurrence of these conditions increases with the length of time NSAIDs are used. (The Physicians' Desk Reference, 56^{th} edition, 2002, p. 2215). These potentially fatal conditions of the GI tract prevent a number of patients from taking NSAIDs, and also reduce the time which any given patient can use NSAIDs.

GI toxicity is not the only problem caused by NSAIDs. They cannot be taken during pregnancy because they may cause premature closure of the ductus arteriosus. There have been cases of severe hepatic reactions, including jaundice and fatal fulminant hepatitis, liver necrosis, and hepatic failure. Long-term administration of NSAIDs has resulted in renal injury, including renal papillary necrosis, and NSAIDs have also been linked to anemia, fluid retention and edema.

In 1999, the new generation of more specific acting NSAIDs, celecoxib and rofecoxib were introduced to solve the problem of COX-1 inhibition. These drugs, known as Celebrex® (U.S. Pat. No. 5,466,823, issued Nov. 14, 1995) and Vioxx® (U.S. Pat. No. 5,474,995, issued Dec. 12, 1995), respectively, specifically target COX-2, and not COX-1. Although they significantly reduce GI tract toxicity, they do not eliminate it completely. Between June 2000 and June 2001, over 100 million prescriptions were issued for Celebrex® and Vioxx®. (JAMA vol. 286 No. 8, p. 954) But, many prescriptions for these drugs still result in the severe GI toxicity in the patients.

Selective COX-2 inhibitors are not without their own set of problems. COX-2 inhibitors such as Celebrex® and Vioxx®, while reducing GI toxicity, have been shown to decrease vascular prostacyclin production, increasing cardiovascular thrombotic events. Cardiovascular thrombotic events can be reduced by the ingestion of aspirin, but this negates the reduction in GI toxicity, and therefore the benefit of the selective COX-2 inhibitors. COX inhibitors, on the other hand, have not been shown to increase cardiovascular thrombotic events, but do increase GI toxicity. Given the trade-off with COX and selective COX-2 inhibitors between GI toxicity and cardiovascular thrombotic events, in addition to the other side effects, it can be hard to choose which drug, if any, to take. A third option is needed. There is a significant need for a safe, effective delivery method for NSAIDs, which will circumvent the GI tract so as not cause GI toxicity in the patient, will not cause the cardiovascular thrombotic events associated with selective COX-2 inhibitors, and will not affect liver and renal function. The present invention teaches a method for transdermal delivery of NSAIDs which achieves all of these goals.

Vioxx®, the chemical formula for which is 4-[4-(methyl-sulfonyl)phenyl]-3-phenyl-2(5H)-furanone, is an NSAID which has been shown to significantly reduce joint pain in individuals with osteoarthritis. It is sparingly soluble in acetone, slightly soluble in methanol and isopropyl acetate, very slightly soluble in ethanol, practically insoluble in octanol, and insoluble in water. (PDR) Vioxx causes the GI toxicity of other NSAIDs, although it is not supposed to inhibit COX-1. Other side effects common to NSAIDs are also caused by Vioxx®.

Celebrex®, with chemical formula 4-[5-(4-methylphenyl)-3-(triflouromethyl)-1 H-pyrazol-1-yl] benzenesulfonamide, is also an NSAID used to treat both osteoarthritis and rheumatoid arthritis. Like all NSAIDs, it can cause severe GI tract toxicity in patients, liver, and kidney damage, with possible fatal consequences. Celebrex, too has a low solubility in aqueous media, but can be delivered transdermally when combined with an isotonic solution as described in the current invention.

The present invention is used to transdermally deliver Vioxx® and Celebrex® to aching joints of a patient, thereby bypassing the GI tract and avoiding any GI toxicity associated with the drug. It results in lower dosages to a specific portion of the body, as opposed to high, system-wide dosages. This delivery method also reduces liver and kidney exposure because of the lower dosage which is significantly diluted by the time it reaches those organs.

The present invention is a method for delivering drugs transdermally, especially insoluble drugs. The powdered form of a drug, such as that contained in the capsules of Vioxx® and Celebrex®, was mixed with Ocean® to form a mixture. The mixture was then rubbed into the skin at the needed location, for example, an arthritic joint, by volunteer subjects suffering from arthritis in different joints. The Vioxx® or Celebrex® suspension in Ocean® then penetrated the outer layer of the skin, traveled through the stratum corneum, dermis, and hypodermis, and entered the target tissue. Within minutes of application by rubbing into the skin closest to the affected arthritic joint, the pain and symptoms are reduced and the subject experiences relief. Therefore, the present invention provides a method of delivery for NSAIDs, such as Vioxx® and Celebrex®, that can inhibit COX-2 at the inflammation site, without ever entering the GI tract, relieving the patient of any GI toxicity that may be caused by the drug.

This method of drug delivery requires lower doses than oral delivery because it goes directly to the target tissue, without being diluted in the blood and delivered throughout the entire body. Since the drug avoids both the gastrointestinal system and the blood stream, side effects are reduced, if not eliminated. Additionally, the amount of the drug which may eventually pass through the kidneys is so low that it will not cause renal damage.

In summary, the method of this patent for drug delivery has been tried by a few patients with severe arthritis. These patients were unable to continue using either Vioxx® or Celebrex® because of the severe GI toxicity those drugs caused. The patients opened the capsules of the drugs, mixed the powdered drug with Ocean®, and applied the solution to the skin of the arthritic area. All patients reported feeling relief from their arthritis without the side effects normally encountered when taking Vioxx® or Celebrex®.

### Accutane®

Just as Vioxx® and Celebrex® can be delivered via the method of the present invention to avoid adverse side effects, so too can Accutane® (U.S. Pat. No. 4,464,394, Expired Feb. 2, 2002). Accutane®, also called isotretinoin, is a retinoid used to treat severe acne by oral delivery of the drug. The actual mechanism of the drug is unknown. Accutane® has numerous severe side effects. Accutane® can cause severe birth defects in children if the drug is taken during pregnancy, or if a woman becomes pregnant within one month of taking Accutane®. Accutane® has also been linked to depression, psychosis, and suicide. In some cases, Accutane® can cause pseudotumor cerebri, (benign intracranial hypertension) or acute pancreatitis. Rare instances of fatal hemorrhagic pancreatitis has also been reported. Accutane® may also cause an elevation in serum triglycerides, including cholesterol. According to the PDR, the affect of elevated lipid levels on the cardiovascular system are unknown. Also linked to Accutane® are clinical hepatitis, inflammatory bowel disease, skeletal hyperostosis and calcification of ligaments and tendons, spontaneous epiphyseal closure, and vision impairment such as corneal opacities and decreased night vision. Therefore, transdermal application of Accutane® directly to the area of acne would require lower dosages, reducing the levels of the drug in the blood stream. It is believed that this would, in turn, reduce, if not eliminate, the harmful side effects associated with Accutane® use. The present invention provides an Accutane® solution in Ocean®, that is suitable for topical application to the affected acne site.

### Carafate®

Yet another drug which can be delivered transdermally using the method of the present invention is Carafate®. Carafate® tablets contain one gram of sucralfate, which is an α-D-glucopyranoside, β-D-frucofuranosyl-, octakis-(hydrogen sulfate), aluminum complex. The Carafate® suspension contains one gram of sucralfate per 10 mL. Carafate® is used to treat duodenal ulcers. It appears that the sucralfate forms an ulcer adherent complex with proteinacious exudates at the ulcer site. The sucralfate-albumin film provides a barrier to diffusion of hydrogen ions, and the sucralfate also inhibits pepsin activity.

Carafate®, surprisingly, can also be used to treat other conditions, including but not limited to acne, herpes simplex II cold sores, root canal wounds, and dry sockets after teeth have been removed. It is believed that Carafate® possesses some anti-inflammatory, antibiotic, antiviral and analgesic characteristics. Since most ulcers are caused by bacteria, it is logical that Carafate®, an ulcer drug, would possess some antibiotic properties, though none has been described before.

Wounds in the mouth are especially prone to infection because of the dark, moist environment, the availability of energy sources from the food that is eaten, and the large numbers of microbes in the mouth. After a root canal or removal of a tooth, a patient is especially prone to infection since the open wound is an idea location for bacteria to settle, and because such a wound is hard to clean. In a number of dental patients, application of Carafate® to root canal wounds or dry sockets after teeth have been removed inhibits infection of those areas, which in turn hastens the healing process and eliminates the need for large doses of antibiotics after such a procedure. Carafate® also seems to reduce pain and swelling in such wounds, so that large doses of pain medications are also not needed.

Herpes Simplex II, more commonly known as genital herpes, is a DNA virus which invades local nerve endings and is transported to the ganglia where it replicates and then remains latent for the lifetime of the infected person. Outbreaks of symptoms occur periodically in response to stimuli such as physical or mental stress, fever, or exposure to ultraviolet light. An outbreak of the disease is often evidenced by cold sores at the corners of the mouth. The cold sores, in addition to being unsightly, can also be painful. However, Carafate® mixed in Ocean® and applied to the cold sores can induce healing of the sores and reduce pain and inflammation. Herpes Simplex I, similar to Herpes Simplex II in many ways, sometimes causes outbreaks of acne. Carafate® mixed with Ocean® and applied to the acne, as taught by the present invention, can heal the outbreak.

What is claimed is:

1. A method for the transdermal delivery of insoluble drugs to a target tissue comprising the steps of:
(a) Mixing a powdered form of an insoluble drug with an isotonic solution to form a suspension;
(b) Spreading said suspension onto an area of skin surrounding said target tissue; and
(c) Rubbing said suspension into said area of skin.

2. The method of claim 1 wherein said isotonic solution is aqueous.

3. The method of claim 2 wherein said aqueous isotonic solution is Ocean®.

4. The method of claim 1 wherein said insoluble drug produces a severe side effect.

5. The method of claim 4 wherein said severe side effect is Gastrointestinal Toxicity.

6. The method of claim 1 wherein said insoluble drug is a Nonsteroidal anti-inflammatory drug (NSAID).

7. The method of claim 6 wherein said NSAID is Celebrex®.

8. The method of claim 6, wherein said NSAID is Vioxx®.

8. The method of claim 1, wherein said insoluble drug is Accutane®.

9. The method of claim 1, wherein said insoluble drug is Carafate®.

10. A method for using the drug Carafate® according to claim 1, wherein the Carafate® is applied to a wound as an antiinflammatory, analgesic, antibiotic, or antiviral.

11. The method of claim 10, wherein said wound is oral.

12. The method of claim 11, wherein said oral wound includes dry socket and root canal wound.

13. The method of claim 11, wherein said wound is acne or a cold sore.

14. The method of claim 13, wherein said acne or cold sore is caused by a Herpes Virus.

15. A composition including one or more pharmaceutical agents in an isotonic solution.

16. The composition as in claim 15, wherein the pharmaceutical agent is selected from the group consisting of agents having toxic adverse effects when taken orally.

17. The composition of claim 15, wherein the pharmaceutical agent is selected form the group of nonsteroidal anti-inflammatory drugs.

18. The composition of claim 15, wherein said pharmaceutical agent is Vioxx®.

19. The composition of claim 15, wherein said pharmaceutical agent is Celebrex®.

20. The composition of claim 15, wherein said pharmaceutical agent is Accutane®.

21. The composition of claim 15, wherein said pharmaceutical agent is Carafate®.

22. The composition of claim 21, having a new use in treatment of oral wounds, including root canal wounds and dry sockets.
